(19) United States
(12) Patent Application Publication
(10) Pub. No.: US 2005/0165242 A1
(43) Pub. Date: Jul. 28, 2005

(54) Process for the preparation of amorphous atorvastatin calcium

(75) Inventors: Daqing Che, Brantford (CA); Aaron C. Kinsman, London (CA); Bhaskar Reddy Guntoori, Brantford (CA); K.S. Keshava Murthy, Ancaster (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Apotex Pharmachem Inc.

(21) Appl. No.: 10/771,383

(22) Filed: Feb. 5, 2004

(30) Foreign Application Priority Data
Jan. 28, 2004 (CA) 2,456,430

(51) Int. Cl. C07D 27/333

(52) U.S. Cl. 548/537

(57) ABSTRACT

A process for the preparation of amorphous atorvastatin calcium and hydrates thereof, which comprises: (a) hydrolysis of the precursor lactone using sodium hydroxide to form atorvastatin sodium salt solution; (b) addition of the atorvastatin sodium salt solution to a calcium chloride or calcium acetate solution in the absence or presence of seeds of amorphous atorvastatin calcium; and (c) isolation of the resultant amorphous atorvastatin calcium salt by filtration and drying.

### FIELD OF INVENTION

An improved industrial process for the preparation of amorphous atorvastatin calcium and hydrates thereof, comprising:
(a) hydrolysis of the precursor atorvastatin lactone using sodium hydroxide to form an atorvastatin sodium salt solution;
(b) addition of the atorvastatin sodium salt solution to a calcium chloride or calcium acetate solution in the absence or presence of amorphous atorvastatin calcium seeds; and
(c) isolation of the resultant amorphous atorvastatin calcium salt by filtration and drying.

### BACKGROUND OF THE INVENTION

Atorvastatin is a reductase inhibitor of the enzyme 3-hydroxy-3-methylglutarate-coenzyme A (HMG-CoA) and therefore is a useful anti-hyperlipoproteinemic agent. It has proven to be a highly effective medicament for the treatment of disorders such as hyperlipidemia and hypercholesterolemia which are conditions that are known risk factors for arteriosclerosis and coronary heart disease. Atorvastatin is chemically [R-(R*,R*)]-2-(4-fluorophenyl)-β,δ-dihydroxy-5-(1-methylethyl)-3-phenyl-4-(phenylcarbamoyl)-1H-pyrrole-1-heptanoic acid and is marketed as its calcium salt under the brand name Lipitor™.

The inhibition of the biosynthesis of cholesterol by the R isomer of atorvastatin was purportedly reported in U.S. Pat. No. 5,273,995. In this patent, it was indicated by the patentee that the calcium salt form of the ring-opened lactone was most effective in terms of formulation. The structure is depicted below as Formula I.

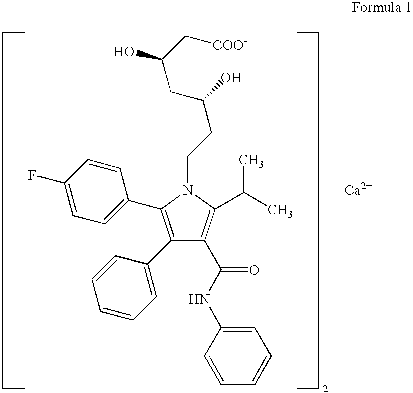

Processes for the manufacture of atorvastatin and key synthetic intermediates have been described in various patents including U.S. Pat. Nos. 4,681,893, 5,003,080, 5,097,045, 5,103,024, 5,124,482, 5,149,837, 5,155,251, 5,216,174, 5,245,047, 5,248,793, 5,280,126, 5,397,792 and 5,342,952. Typically, the final stages of the process involve the conversion of the precursor lactone [(2R-trans)-5-(4-fluorophenyl)-2-(1-methylethyl)-N,4-diphenyl-1-[2-(tetrahydro-4-hydroxy-6-oxo-2H-pyran-2-yl)ethyl]-1H-pyrrole-3-carboxamide, formula II, referred herein as atorvastatin lactone] to the corresponding sodium carboxylate salt by base hydrolysis.

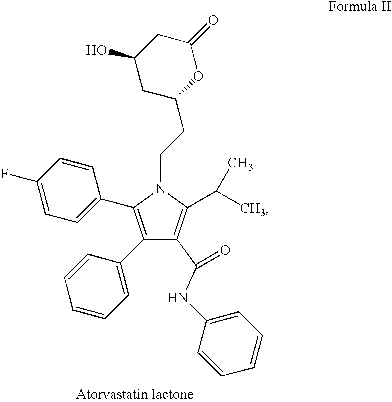

However, the processes described in the patents mentioned above do not consistently yield atorvastatin calcium in the amorphous form where the resulting product has suitable filtration and drying properties. These deficiencies pose difficulties in terms of large-scale production of amorphous atorvastatin calcium.

Disclosed in U.S. Pat. No. 5,969,156 are novel crystalline forms of atorvastatin calcium, which are designated as Form I, Form II and Form IV. Also disclosed are methods for their preparation. Amorphous atorvastatin calcium has advantages relative to the crystalline form, for instance in terms of dissolution and, in some cases, bioavailability.

A procedure for the conversion of the crystalline form of atorvastatin to the amorphous form is described in U.S. Pat. No. 6,274,740 B1. This process involves dissolution of crystalline Form I atorvastatin calcium in a non-hydroxylic solvent such as tetrahydrofuran and/or toluene. The process suffers from the deficiency that the solvent must be completely removed under vigorous conditions; namely, at high temperature (ca. 85° C.) and high vacuum. This requires the use of specialized and expensive equipment. Furthermore, the exposure of atorvastatin to high temperatures for prolonged periods (4 days), for instance as described in example 2 of U.S. Pat. No. 6,087,511 and U.S. Pat. No. 6,274,740 B1, may cause product degradation. Finally, the solvents used for this process are undesirable in terms of toxicity. All of these factors combine to make this process difficult for further scale-up.

Other processes are described for the production of amorphous atorvastatin calcium, for instance in U.S. Pat. No. 6,528,660 B1, U.S. Pat. No. 6,613,916 B2, U.S. Pat. No. 6,646,133 B1 and WO 03/078379. However all of these suffer from the fact they either involve dilute reaction conditions, use undesirable solvents and/or begin from the atorvastatin calcium. They rely on precipitation of the amorphous atorvastatin calcium or a spray drying procedure for isolation.

WO 02/083638 does begin from atorvastatin sodium and teaches a similar process to the one described in U.S. Pat. No. 5,969,156 where the aqueous calcium chloride solution is added to the atorvastatin sodium solution.

WO 03/068739 also does begin from atorvastatin sodium, however a rather lengthy work-up procedure is required in addition to the use of co-solvents such as toluene, methyl tert-butyl ether, pentane and tetrahydrofuran.

One object of the present invention is to overcome the deficiencies of the prior art thereby allowing an efficient, scalable, cost-effective and robust process to produce amorphous atorvastatin, directly from the atorvastatin lactone.

Further and other objects of the invention will be realized by those skilled in the art from the following Summary of the Invention and Detailed Description of Embodiments thereof.

### SUMMARY OF THE INVENTION

According to one aspect of the invention, there is provided a process for producing amorphous atorvastatin calcium comprises:
(a) hydrolysis of atorvastatin lactone (formula II) with sodium hydroxide to form atorvastatin sodium salt solution;
(b) addition of the atorvastatin sodium salt solution to an aqueous calcium chloride or calcium acetate solution; and
(c) isolation by filtration and drying affording amorphous atorvastatin calcium salt.

According to another aspect of the invention, there is provided a process for producing amorphous atorvastatin calcium comprises:
(a) hydrolysis of atorvastatin lactone (formula II) to form an atorvastatin salt solution;
(b) addition of the atorvastatin salt solution to an aqueous calcium salt solution; and
(c) isolation by filtration and drying affording amorphous atorvastatin calcium salt.

Surprisingly, contrary to WO 02/083638, we have discovered that the order of addition in step (b) is important in terms of the morphology of the resulting amorphous atorvastatin calcium. The order of addition in step (b) as per the present invention results in isolation of a product, which is significantly easier to filter and dry. Furthermore, the filter cake obtained when following the procedure of the present invention retains less residual water relative to the standard mode of addition. For instance, the water content of the filter cake using the conventional order of addition is about 10% to 20% higher relative to the present invention. These differences are of importance when transiting to larger scale manufacture of amorphous atorvastatin calcium salt.

According to another aspect of this invention, the process may comprise the addition of seeds of amorphous atorvastatin calcium to the calcium chloride or calcium acetate solution of step (b).

Some unexpected advantages of the present invention (with or without seeding with amorphous atorvastatin calcium), relative to the prior art include but are not limited to:
(a) elimination of the solvent removal step after atorvastatin lactone hydrolysis,
(b) faster filtration of the amorphous atorvastatin calcium
(c) robust and scalable process amenable to industrial production,
(d) produces amorphous atorvastatin calcium having low residual solvent levels including water and other solvents which is valuable given the high purity specification required in the pharmaceutical industry,
(e) safer, industrially acceptable solvents used throughout (water and methanol), relative to prior art methods, and
(f) improved stability.

According to another aspect of the invention, there is provided amorphous atorvastatin calcium preferably which contains at least one of the following:
(i) residual amounts of water,
(ii) residual amounts of solvent other than water.

Preferably, in the process according to the present invention, the hydrolysis of atorvastatin lactone of formula II is accomplished using sodium hydroxide.

Preferably, in the process the solution of atorvastatin sodium in water and methanol is added to a solution of calcium chloride or calcium acetate in water containing seeds of amorphous atorvastatin calcium.

Preferably, the quantity of seeds of amorphous atorvastatin calcium is in the range of from about 0.05 to about 10 weight percent relative to the atorvastatin lactone, more preferably, in the range of from about 0.1 to about 5 weight percent relative to the atorvastatin lactone, even more preferably it is about 0.2 weight percent relative to the atorvastatin lactone.

In another embodiment the solution of atorvastatin sodium in water and methanol is added to a solution of calcium chloride or calcium acetate in water without seeds of amorphous atorvastatin calcium.

Preferably, in the process the stoichiometry of the sodium hydroxide relative to atorvastatin lactone is from about 0.85 to about 1.05 equivalents. More preferably, the stoichiometry of the sodium hydroxide relative to atorvastatin lactone is from about 0.9 to about 1.0 equivalents. Even more preferably, the stoichiometry of the sodium hydroxide relative to atorvastatin lactone is about 0.98 equivalents.

Preferably, the stoichiometry of calcium chloride or calcium acetate relative to atorvastatin lactone is from about 0.4 to 1.5 equivalents. More preferably, the stoichiometry of calcium chloride or calcium acetate relative to atorvastatin lactone is from about 0.45 to 0.55 equivalents. Even more preferably, the stoichiometry of calcium chloride or calcium acetate relative to atorvastatin lactone is about 0.5 equivalents.

Preferably, the hydrolysis reaction requires from about 1 to 24 hours. More preferably, the hydrolysis reaction requires from about 10 to 20 hours. Even more preferably, the hydrolysis reaction requires from about 12 to 14 hours.

Furthermore, there is provided amorphous atorvastatin calcium substantially free of residual solvents.

Furthermore, the process herein results in the product substantially free of residual solvents.

Furthermore, there is provided the use of amorphous atorvastatin calcium substantially free of residual solvents in the manufacture of a pharmaceutical composition for treating hypercholesterolemia.

Furthermore, there is provided for use in inhibiting cholesterol synthesis in a human suffering from hypercholesterolemia, a compound of the process descried herein.

Furthermore, there is provided the use of amorphous atorvastatin calcium substantially free of residual solvents in the treatment of hypercholesterolemia.

In one embodiment of the invention atorvastatin lactone of formula II is hydrolyzed in volumes from about 3 to about 10 volumes (relative to atorvastatin lactone) of a solution of methanol and water preferably in a ratio from about 1:1 to about 15:1, more preferably from about 3:1 to about 10:1, and most preferably 5:1 (v/v). The hydrolysis is performed preferably at a temperature ranging from about 10° C. to about 30° C., more preferably from about 15° C. to about 25° C. using from about 0.85 to about 1.05 equivalents of sodium hydroxide, more preferably, about 0.9 to about 1.0 equivalents, and most preferably about 0.98 equivalents. The hydrolysis reaction requires from about 1 to about 24 hours, more preferably from about 10 to about 20 hours, and most preferably from about 12 to about 14 hours.

After completion of hydrolysis, the atorvastatin sodium salt solution is washed with at least one organic solvent or mixtures thereof, preferably ethyl acetate and heptane mixtures. This accomplishes the removal of atorvastatin lactone and other unwanted impurities. The washed atorvastatin sodium salt solution is then added to an aqueous calcium solution preferably an aqueous calcium solution of calcium chloride or calcium acetate. The volume of the aqueous calcium solution required is one which allows for sufficient agitation, which is about 2 to 10 volumes. The most preferable stoichiometry of calcium chloride or calcium acetate is from about 0.4 to about 1.5 equivalents (relative to the atorvastatin lactone starting material), more preferably from about 0.45 to about 0.55 equivalents, and most preferably about 0.50 equivalents. The calcium salt formation is conducted at about 0° C. to about 60° C., more preferably at about 15° C. to about 50° C., and most preferably at about 20° C. to about 25° C. In a preferred embodiment, aqueous solution of calcium chloride or calcium acetate may contain seeds of amorphous atorvastatin calcium at a weight percentage of from about 0.05% to about 10%, more preferably from about 0.1% to about 5%, most preferably about 0.2%. Washing of the formed atorvastatin calcium with a solvent, such as water accomplishes the removal of any unwanted sodium salt in the final product.

### DETAILED DESCRIPTION OF EMBODIMENTS

### REFERENCE EXAMPLE 1

Conventional Formation of Atorvastatin-Ca:

To a slurry of (2R-trans)-5-(4-fluorophenyl)-2-(1-methylethyl)-N,4-diphenyl-1-[2-(tetrahydro-4-hydroxy-6-oxo-2H-pyran-2-yl)ethyl]-1H-pyrrole-3-carboxamide (9.60 g, 17.76 mmol) in methanol (48 mL) and water (8 mL) was added sodium hydroxide (0.69 g, 17.2 mmol). After hydrolysis was complete, the mixture was diluted with water (40 mL) and washed twice with ethyl acetate/heptane (48 mL; 1:1). An aqueous solution (40 mL) of CaCl₂ (0.99 g, 8.92 mmol) was then added to the sodium salt solution. The resulting solid was Buchner filtered and washed with 20 mL water and the damp cake (35.9 g) was dried under vacuum to afford amorphous atorvastatin calcium (8.60 g, 82% yield). The material was characterized as amorphous atorvastatin calcium based on its powder X-Ray diffraction pattern and DSC.

### EXAMPLE 2

Formation of Atorvastatin-Ca by Inverse Addition Without Seeding:

To a slurry of (2R-trans)-5-(4-fluorophenyl)-2-(1-methylethyl)-N,4-diphenyl-1-[2-(tetrahydro-4-hydroxy-6-oxo-2H-pyran-2-yl)ethyl]-1H-pyrrole-3-carboxamide (10.20 g, 19.0 mmol) in methanol (51 mL) and water (10 mL) was added sodium hydroxide (0.74 g, 18.4 mmol). After hydrolysis was complete, the mixture was diluted with water (42 mL) and washed twice with ethyl acetate/heptane (51 mL; 1:1). The sodium salt solution was then added to an aqueous solution (40 mL) of CaCl₂ (1.05 g, 9.50 mmol). The resulting solid was Buchner filtered and washed with 20 mL water and the damp cake (24.9 g) was dried to afford amorphous atorvastatin calcium (8.05 g, 77% yield). The material was characterized as amorphous atorvastatin calcium based on its powder X-Ray diffraction pattern and DSC. The combined time required for the filtration and washing steps when carried out under the same filtration and washing conditions as example 1, was reduced by 71%, relative to example 1. Likewise, the moisture content of the damp filter cake of example 2 was about 12% less, relative to example 1.

### EXAMPLE 3

Formation of Atorvastatin-Ca by Inverse Addition with Seeding:

To a slurry of (2R-trans)-5-(4-fluorophenyl)-2-(1-methylethyl)-N,4-diphenyl-1-[2-(tetrahydro-4-hydroxy-6-oxo-2H-pyran-2-yl)ethyl]-1H-pyrrole-3-carboxamide (10.40 g, 19.24 mmol) in methanol (52 mL) and water (10 mL) was added NaOH (0.74 g, 18.6 mmol). After hydrolysis was complete, the mixture was diluted with water (42 mL) and washed twice with ethyl acetate/heptane (52 mL; 1:1). The sodium salt solution was then added to a suspension of amorphous atorvastatin calcium seed (0.02 g) in an aqueous solution (40 mL) of CaCl₂ (1.06 g, 9.55 mmol). The resulting solid was Buchner filtered and washed with 20 ml water and the damp cake (22.2 g) was dried under vacuum to afford amorphous atorvastatin calcium (8.91 g, 86% yield). The material was characterized as amorphous atorvastatin calcium based on its powder X-Ray diffraction pattern and DSC. The combined time required for the filtration and washing steps when carried out under the same filtration and washing conditions as example 1 was reduced by 53%, relative to example 1. Likewise, the moisture content of the damp filter cake of example 3 was about 20% less, relative to example 1.

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and not in a limiting sense.

1. A process for the preparation of amorphous atorvastatin calcium which comprises:
(a) hydrolysis of the atorvastatin lactone of formula II to form atorvastatin sodium salt solution;
(b) addition of the atorvastatin sodium salt solution to an aqueous calcium chloride or calcium acetate solution; and
(c) isolation by filtration and drying to afford amorphous atorvastatin calcium salt.

2. The process of claim 1 wherein the amorphous atorvastatin calcium contains at least one of the following:
(i) residual amounts of water,
(ii) residual amounts of solvent other than water.

3. The process of claim 1 or 2, wherein the hydrolysis of atorvastatin lactone of formula II is accomplished using sodium hydroxide, resulting in an atorvastatin sodium salt solution.

4. The process of any one of claims 1 to 3, wherein the solution of atorvastatin sodium in water and methanol is added to a solution of calcium chloride or calcium acetate in water containing seeds of amorphous atorvastatin calcium.

5. The process of claim 4 wherein the quantity of seeds of amorphous atorvastatin calcium is in the range of from about 0.05 to about 10 weight percent relative to the atorvastatin lactone.

6. The process of claim 5 where the quantity of seeds of amorphous atorvastatin calcium is in the range of from about 0.1 to about 5 weight percent relative to the atorvastatin lactone.

7. The process of claim 6 where the quantity of seeds of amorphous atorvastatin calcium is in the range of from about 0.2 weight percent relative to the atorvastatin lactone.

8. The process of any one of claims 1 to 3, wherein the solution of atorvastatin sodium in water and methanol is added to a solution of calcium chloride or calcium acetate in water without seeds of amorphous atorvastatin calcium.

9. The process of claim 3, wherein the stoichiometry of the sodium hydroxide relative to atorvastatin lactone is from about 0.85 to about 1.05 equivalents.

10. The process of claim 3, wherein the stoichiometry of the sodium hydroxide relative to atorvastatin lactone is from about 0.9 to about 1.0 equivalents.

11. The process of claim 3, wherein the stoichiometry of the sodium hydroxide relative to atorvastatin lactone is about 0.98 equivalents.

12. The process of claim 1 or 2 where the stoichiometry of calcium chloride or calcium acetate relative to atorvastatin lactone is from about 0.4 to 1.5 equivalents.

13. The process of claim 1 or 2 where the stoichiometry of calcium chloride or calcium acetate relative to atorvastatin lactone is from about 0.45 to 0.55 equivalents.

14. The process of claim 1 or 2 where the stoichiometry of calcium chloride or calcium acetate relative to atorvastatin lactone is from about 0.5 equivalents.

15. The process of claim 1 or 2 wherein the hydrolysis reaction requires from about 1 to 24 hours.

16. The process of claim 1 or 2 wherein the hydrolysis reaction requires from about 10 to 20 hours.

17. The process of claim 1 or 2 wherein the hydrolysis reaction requires from about 12 to 14 hours.

18. Amorphous atorvastatin calcium substantially free of residual solvents.

19. The process of any of claims 1, 2, 3, 4 or 8 wherein the product is substantially free of residual solvents.

20. The use of amorphous atorvastatin calcium substantially free of residual solvents in the manufacture of a pharmaceutical composition for treating hypercholesterolemia.

21. For use in inhibiting cholesterol synthesis in a human suffering from hypercholesterolemia, a compound of claim 18.

22. The compound of claim 18 wherein the residual solvents are selected from water and methanol.

23. A process for the preparation of amorphous atorvastatin calcium which comprises:
(a) hydrolysis of the atorvastatin lactone of formula II to form atorvastatin salt solution;
(b) addition of the atorvastatin salt solution to an aqueous calcium salt solution; and
(c) isolation by filtration and drying to afford amorphous atorvastatin calcium salt.

24. Use of amorphous atorvastatin calcium substantially free of residual solvents in the treatment of hypercholesterolemia.
